(19) United States
(12) Patent Application Publication
(10) Pub. No.: US 2008/0188932 A1
(43) Pub. Date: Aug. 7, 2008

(54) COATED MEDICAL IMPLANTS, METHODS OF COATING MEDICAL IMPLANTS, AND METHODS OF COATING MATERIALS

(76) Inventors: Mason K. Harrup, Idaho Falls, ID (US); Frederick F. Stewart, Idaho Falls, ID (US); Thomas A. Luther, Idaho Falls, ID (US)

Correspondence Address: BATTELLE ENERGY ALLIANCE, LLC, P.O. BOX 1625, IDAHO FALLS, ID, 83415-3899, US

(21) Appl. No.: 11/672,409

(22) Filed: Feb. 7, 2007

(51) Int. Cl. A61L 33/06; A61F 2/02

(52) U.S. Cl. 623/11.11; 427/2.24

(57) ABSTRACT

Embodiments include polymeric coatings formed from poly((perfluoro)organo)phosphazene-based materials. The coatings may be utilized to coat any suitable substrates, and in some embodiments may be utilized to coat surfaces of medical implants. The poly((perfluoro)organo)phosphazene-based materials may contain some fluorine-containing groups, and some crosslinkable moieties. The poly((perfluoro)organo)phosphazene-based materials may also contain some groups suitable for covalent bonding with surfaces of particular substrates. For instance, the poly((perfluoro)organo)phosphazene-based materials may include amine-containing groups for covalently bonding to urethane surfaces.

### GOVERNMENT RIGHTS

The United States Government has certain rights in this invention pursuant to Contract No. DE-AC07-05ID14517 between the United States Department of Energy and Battelle Energy Alliance, LLC.

### TECHNICAL FIELD

The invention pertains to coated medical implants, methods of coating medical implants, and methods of coating materials.

### BACKGROUND

Each year, millions of medical implants made of synthetic materials are used on human patients. The vast majority of these are in contact with the blood. The blood may stick to the implants and create blood clots. Accordingly, the patients generally take blood-thinning medicines (called anticoagulants) after receiving the implants, and for the duration of time that the implants are in their bodies. The patients also typically undergo frequent bloodwork to monitor the efficacy of the blood-thinning medicines. Bleeding due to an anticoagulant regimen is a significant threat to the patients, but other problems are also generated, such as thrombosis, thromboembolism, and peripheral ischemic complications, including stroke.

Another aspect of the prior art is that millions of people are diagnosed with heart valve disease each year. Heart valve disease may result from valve degeneration caused by calcification, by weakened supportive structures in the valve, by infections (such as rheumatic valve disease or bacterial infections), by congenital abnormalities. and/or by damage from medications (such as Phen-Fen).

Treatment for heart valve disease may include surgery to repair or replace defective valves. The success rate for heart valve surgery is good. However, complications can arise. Such complications may include thrombosis, thromboembolism, and/or other complications from coagulation and anticoagulation drug regimens.

Thrombotic and hemorrhagic complications are a major cause of morbidity and mortality, and therefore are important determinants of long-term outcome following mechanical valve surgery. Thrombosis on prosthetic valves may give rise to local mechanical problems, including valve obstruction. It may also lead to thromboembolism and/or peripheral ischemic complications, which may include stroke. Research continues to improve surgical techniques, and to reduce the thrombogenicity of replacement heart valves; but the risk of thrombosis, thromboembolism, and bleeding remains.

It is desired to develop improvements which can increase safety for patients receiving medical implants.

### SUMMARY OF THE INVENTION

In some embodiments, a polymeric coating is formed from poly((perfluoro)organo)phosphazene-based polymer, which may be heteropolymer. The coating can be utilized to coat any suitable substrate. In some aspects, the coating is utilized to coat surfaces of medical implants.

The poly((perfluoro)organo)phosphazene-based heteropolymers may comprise short-chain fluorine-containing groups for biocompatibility, and may contain some groups having readily crosslinkable moieties. The cross-linkable moieties may be provided to be of suitable type and relative concentration to create desired mechanical properties of the coating. In some applications, poly((perfluoro)organo)phosphazene-based heteropolymers may also contain groups suitable for covalent bonding with a surface of particular substrate.

In some embodiments, the invention includes coated medical implants. An example embodiment coated medical implant is a medical implant at least partially coated with polymeric material comprising a subunit which includes:

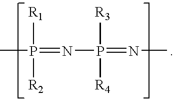

The groups R₁, R₂, R₃ and R₄ are carbon-containing groups, two or more of which may be the same as one another. At least one of R₁, R₂, R₃ and R₄ comprises —(CF₂)ₙ—CF₂(H,F), where n is an integer greater than or equal to 1. At least one of R₁, R₂, R₃ and R₄ of one subunit may be directly cross-linked to an R₁, R₂, R₃ or R₄ of another subunit, or may be cross-linked to one of the other of the R₁, R₂, R₃ and R₄ of the same subunit. If R₁, R₂, R₃ or R₄ of one subunit is cross-linked to another subunit, such other subunit may be part of a polymer chain comprising said one subunit, or may be part of a different polymer chain than that comprising said one subunit.

The polymer containing the above-described subunit may comprise randomly substituted subunits, and accordingly the described subunit may be one of many different types of subunits present in the polymer. In some applications, the subunit will be a predominate subunit of the polymer.

In some embodiments, the invention includes methods of coating medical implants. Surfaces of the implants may be exposed to a composition comprising material containing a subunit which includes:

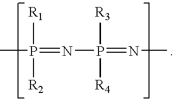

The groups R₁, R₂, R₃ and R₄ are carbon-containing groups. At least one of R₁, R₂, R₃ and R₄ comprises —(CF₂)ₙ—CF₂(H,F) , where n is an integer greater than or equal to 1. At least one of R₁, R₂, R₃ and R₄ comprises an ω-olefin. Two or more of R₁, R₂, R₃ and R₄ may be the same as one another. The material may comprise randomly substituted subunits, and accordingly the described subunit may be one of many different types of subunits present in the material. In some applications, the subunit will be a predominate subunit of the material. The composition is formed into a polymeric coating adhered to the implant.

In some embodiments, the invention includes methods of coating substrates. The substrates may be exposed to material containing a subunit of:

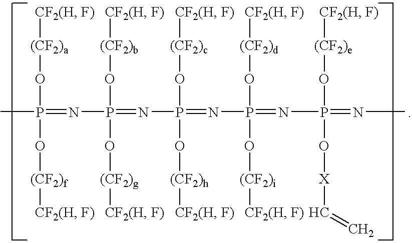

The X may comprise carbon and/or heteroatoms. The a, b, c, d, e, f, g, h and i are integers of from 1 to 16, which may be the same as one another or different. The material may comprise randomly substituted subunits, and accordingly the described subunit may be one of many different types of subunits present in the material. In some applications, the subunit will be a predominate subunit of the material. The material is treated to convert it to a polymeric coating surrounding at least a portion of the substrate.

In some embodiments, the invention includes other methods of coating substrates. The substrates may be exposed to material containing a subunit of:

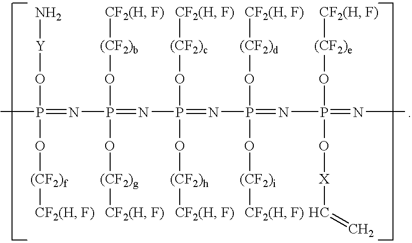

The X comprises carbon and/or heteroatoms. The Y comprises carbon and/or heteroatoms. The b, c, d, e, f, g, h and i are integers of from 1 to 16, which may be the same as one another or different. The material may comprise randomly substituted subunits, and accordingly the described subunit may be one of many different types of subunits present in the material. In some applications, the subunit will be a predominate subunit of the material. The material is treated to convert it to a polymeric coating surrounding at least a portion of the substrate.

### BRIEF DESCRIPTION OF THE DRAWINGS

FIGS. 1 and 2 are a diagrammatic cross-sectional view, and a diagrammatic top view, respectively, of a substrate at a preliminary processing stage in accordance with an aspect of the present invention. The view of FIG. 1 is along the line 1-1 of FIG. 2.

FIG. 3 is a view of the FIG. 1 cross-section shown in an assembly at a processing stage subsequent to that of FIGS. 1 and 2.

FIG. 4 is a view of the substrate of FIG. 1 shown at a processing stage subsequent to that of FIG. 3.

FIGS. 5 and 6 are a diagrammatic cross-sectional view and a diagrammatic top view, respectively, of the substrate of FIGS. 1 and 2 shown at a processing stage subsequent to that of FIG. 4. The cross-section of FIG. 5 is along the line 5-5 of FIG. 6.

### DETAILED DESCRIPTION OF THE ILLUSTRATED EMBODIMENTS

In some aspects, the invention includes a recognition that accumulation of blood platelets onto medical implants is a surface interaction phenomenon, and a further recognition that it would be desirable to develop a modification for surfaces of proven devices to reduce negative interactions. Modification of surfaces of proven clinical devices, as opposed to developing entirely new designs, may be a more expeditious route for providing quality devices to the patients that need them. Coatings of the present invention may also be utilized on yet-to-be developed devices.

Some aspects of the present invention include new biocompatible coatings for medical implants. Such coatings may eliminate or significantly reduce negative interactions between surfaces of the implants and blood components, and may have high durability. For purposes of interpreting this disclosure, the term “medical implant” means any implant surgically inserted into any animal. Accordingly, the term can include dental implants, heart valve implants, skeletal support or replacement implants, arterial support or replacement structures, etc.; can include implants utilized for human medical applications, and can include implants utilized for veterinary applications. The term does not include dentures, as such are not surgically inserted.

Some aspects include utilization of various fluorinated derivatives of the phosphazene polymer family as biocompatible coatings. Poly((perfluoro)organo)phosphazenes can have desired properties for medical implant coatings in that poly((perfluoro)organo)phosphazenes can have blood/tissue compatibility, and can have resistance to surface microbial/fungal incursion.

Poly((perfluoro)organo)phosphazenes can be synthesized by macromolecular nucleophilic substitution reactions. The composition of individual polymer formulations can be controlled to provide specificity for synthesis of desired coatings.

It can be desired that poly((perfluoro)organo)phosphazenes utilized as coatings have sufficient mechanical integrity to substantially avoid delamination from a coated substrate. Delamination can be problematic with any coatings, but can be particularly problematic with coatings of medical implants. Delamination from medical implants can lead to release of delaminated material into the blood, and/or can lead to exposure of substrate material of the medical implants to the blood.

The mechanical properties of medical implants can vary widely. For instance, some implants are rigid sheets (such as mechanical heart valves), and some are flexible tubes (such as artificial arteries).

It can be desired to utilize a general coating chemistry that is suitable for application to a wide array of substrates. In can be particularly desired to utilize coating chemistry that can provide good biocompatibility when utilized with medical implants, and yet which also has mechanical stability when applied to the diverse mechanical structures present within the field of medical implants.

In some embodiments, coatings are formed to address the goals discussed above. The coatings comprise custom poly((perfluoro)organo)phosphazenes which are cured to form desired coatings on a wide array of substrates. The curing may adhere a coating to a substrate. The curing may additionally, or alternatively, form polymer/polymer physical entanglement between polymer chains. The adhering and/or physical entanglement may be through solution/diffusion bonding, direct covalent condensation (as with urethane linkages), or activated covalent bonding (such as radical generation/interfacial covalent bonding). The curing may impart desired mechanical properties to the coating (for example, elasticity, toughness, etc.).

Numerous methods may be utilized to expose a substrate surface to poly((perfluoro)organo)phosphazene material. For instance, spray coating, dip coating, spin casting, and/or static solution casting may be utilized. As the substrate surface is exposed, the material is cured to form a coating adhered to the surface. The curing can be tailored to permanently secure the coating into place, and to provide desired final mechanical properties to the coating. Any suitable technique can be utilized for the curing, including, for example, deep ultraviolet (UV) light radiation crosslinking techniques, and electron beam crosslinking techniques. Other techniques that can be incorporated into the curing are solution blending and composite formulation, which can be particularly useful for integrating the poly((perfluoro)organo)phosphazene coating into bulk core material.

In some applications, a substrate surface may be modified to make it more amenable to coating. This can involve chemical/radiation treatment of the substrate to place reactive chemical groups onto the substrate surface. Example reactive chemical groups are groups suitable for direct condensation with one or more groups present in the poly((perfluoro)organo)phosphazene material.

In some embodiments, a poly((perfluoro)organo)phosphazene having the general formula shown below as Formula I is utilized.

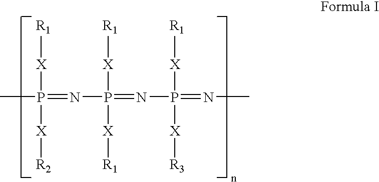

In Formula I, the groups R₁ are short-chain fluorinated alkyl groups; the group R₂ is a hydrocarbon, that may or may not comprise heteroatoms, suitable for forming crosslinks (and may, for example, comprise an unsaturated hydrocarbon, and may specifically comprise an ω-olefinic hydrocarbon in some embodiments); and the group R₃ is a substrate matched interfacial bonding group. The label “X” represents S, N or O. The label “n” is an integer, and can be from 2 to many orders of magnitude.

The poly((perfluoro)organo)phosphazene formula above illustrates principal components. Typically there will be a heterogeneous mixture of polymers. The polymers will be randomly substituted in desired ratios of components as a consequence of the synthetic pathway of macromolecular nucleophilic substitution. Such substitution can be with a terminal hydroxyl functionality, which will lead to “X” being O.

The major component group (R₁) may be comprised of short chain heavily fluorinated alkyl groups. Such fluorinated alkyl groups can impart desired biocompatibility.

The minor component R₂ comprises a hydrocarbon, that may or may not comprise heteroatoms, and may be utilized to accomplish poly((perfluoro)organo)phosphazene/poly((perfluoro)organo)phosphazene crosslinking. For instance R₂ may comprise an alkyl with one or more carbon-hydrogen bonds which can be converted to cross-links with exposure to suitable energy, may comprise an unsaturated hydrocarbon, and in some embodiments may comprise an ω-olefinic hydrocarbon. The cross-linking can allow for physical/mechanical property tailoring within the coating itself, decoupled from other parts of the coating formation.

The minor component R₃ comprises a covalently condensable specie that is amenable for bonding/adhesion interactions to a substrate which is to be coated.

In some embodiments the invention includes methods of coating substrate surfaces with polymeric phosphazene material. The substrate surfaces may be organic, metallic, or ceramic. In some applications, the substrate surfaces are polymeric, and the coating of the substrate surfaces comprises forming covalent bonds to the surfaces. In other applications, covalent bonds are not formed to the substrate surfaces, but instead interlinking between polymeric chains of phosphazene material creates a polymeric mesh tightly bound around a substrate.

The substrates may correspond to any structures desired to be coated with polymeric phosphazene material. The coating may be utilized to protect the substrates, and/or to form barriers between the substrates and other materials. In some applications, the substrates may correspond to medical implants and the polymeric phosphazene material may be a biocompatible coating. The biocompatible coating should be provided across all exposed surfaces of the medical implant. In some embodiments, the biocompatible coating will be provided across any surfaces that could otherwise contact blood or tissue.

An example embodiment for coating a substrate (such as a medical implant) is to expose a surface of the substrate to a poly((perfluoro)organo)phosphazene composition. The composition may contain a subunit which includes Formula II.

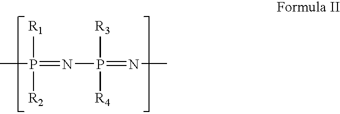

In Formula II, R₁, R₂, R₃ and R₄ are carbon-containing groups. At least one of R₁, R₂, R₃ and R₄ comprises —CF₂—CF₂(H,F). The nomenclature —CF₂(H,F) is used to designate a terminal carbon bonded to two fluorine atoms, and to either another fluorine or a hydrogen. Thus the nomenclature —CF₂(H,F) is equivalent to a Markush designation of —CF₃ or —CHF₂. At least one of R₁, R₂, R₃ and R₄ of Formula II comprises an ω-olefin. The term ω-olefin (omega-olefin) means any terminal double bond between a pair of carbon atoms. Two or more of R₁, R₂, R₃ and R₄ may be the same as one another.

The groups R₁, R₂, R₃ and R₄ may comprise heteroatoms in addition to carbon. Also, the groups may be linear, cyclic or aromatic; or may comprise liner, cyclic or aromatic portions. The poly((perfluoro)organo)phosphazene composition may comprise only subunits corresponding to Formula II, or may comprise other subunits in addition to those corresponding to Formula II.

In some embodiments, the subunit of Formula II may correspond to Formula III.

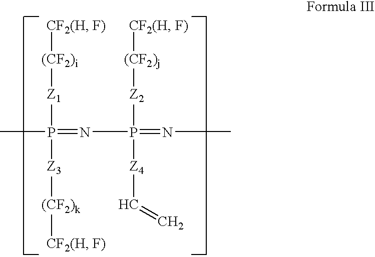

In Formula III, Z₁, Z₂, Z₃, and Z₄ comprise N, S or O, and may be the same as one another or different. The labels i, j and k are integers of from 1 to 16, and may be the same as one another or different. A specific example of a Formula III subunit where Z₁, Z₂, Z₃, and Z₄ comprise O is shown as Formula IV.

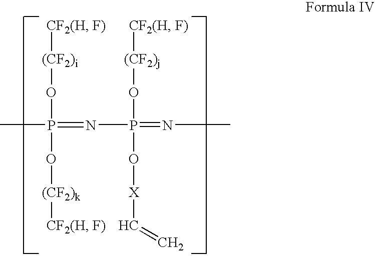

In Formula IV, X comprises one or more of carbon and heteroatoms. In some embodiments, X may comprise —((CH₂)₂O—)ₙ, where n is an integer from 1 to 16. The X may comprise a linear carbon-containing structure, a cyclic carbon-containing structure, and/or an aromatic carbon-containing structure.

The subunit of Formulas II, III and IV may comprise more than the two shown phosphorus groups of the phosphazene chain. For instance, the subunit comprising Formula IV may correspond to Formula V.

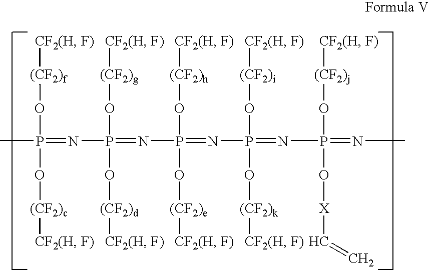

In Formula V; c, d, e, f, g and h are integers of from 1 to 16, which may be the same as one another or different. One or more of c, d, e, f, g and h may be the same as i, j or k, or different.

The formation of a coating from a material comprising one or more of Formulas II, III, IV and V comprises exposure of a substrate surface to the material, followed by curing of the material to convert the material to a polymeric coating. The polymeric coating will comprise chains of phosphazene polymer. Such chains may be linear, cyclolinear or cyclomatrix, (the terms linear, cyclolinear and cyclomatrix, relative to phosphazene polymers, are described in U.S. Pat. No. 6,403,755 (Stewart)). It may be preferred that the chains be predominately, or even entirely, linear.

The ω-olefin of the material comprising one or more of Formulas II, III, IV and V may form cross-linking between polymeric chains of the polymeric coating, and/or may form a covalent bonds to a substrate surface. In some embodiments, a substrate that is to be coated with polymeric material has a relatively non-reactive surface. In such embodiments, the ω-olefin may enable sufficient cross-linking within the polymeric coating to form a tightly interconnected mesh of polymeric material surrounding the substrate. Such mesh may tightly adhere to the substrate in spite of the lack of covalent bonding between the substrate and the polymeric coating, due to the geometric encapsulation of the substrate within the polymeric coating. For instance, in some embodiments medical implant substrates will have relatively non-reactive surfaces of titanium or other metallic materials, and coating compositions comprising one or more of Formulas II, III, IV and V can be utilized to form polymeric phosphazene coatings encapsulating such substrates.

In some embodiments, substrates are utilized which have surfaces that readily react under appropriate conditions to form covalent bonds. The substrates may correspond to commercially-available medical implants, and such surfaces may correspond to surfaces of the substrates as commercially obtained, or may correspond to surfaces modified to become more reactive. The coating material of Formula II may be configured to comprise groups suitable for reacting with the substrate surfaces. For instance, in some embodiments a substrate may comprise a group reactive with amines to form covalent bonds. Such group may comprise, for example, carbonyl suitable for forming amide linkages. The coating material of Formula II may then be configured to comprise a terminal amine, and may, for example, be configured as Formula VI.

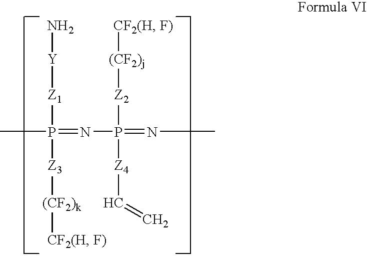

In Formula VI, Z₁, Z₂, Z₃, and Z₄ comprise N, S or O, and may be the same as one another or different; and Y comprises one or more of carbon and heteroatoms. The labels j and k are integers of from 1 to 16; and may be the same as one another or different. A specific example of a Formula VI subunit where Z₁, Z₂, Z₃, and Z₄ comprise O is shown as Formula VII.

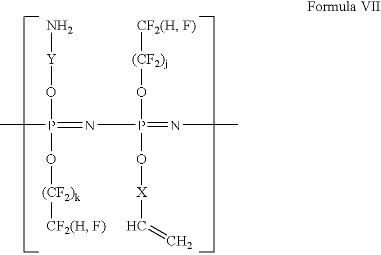

In Formula VII, X comprises one or more of carbon and heteroatoms. In some embodiments, X may comprise —((CH₂)₂O—)ₙ, where n is an integer from 1 to 16. The X may comprise a linear carbon-containing structure, a cyclic carbon-containing structure, and/or an aromatic carbon-containing structure. The Y may comprise —((CH₂)—)ₙ, where n is an integer from 1 to 16. Also, the Y may comprise a linear carbon-containing structure, a cyclic carbon-containing structure, and/or an aromatic carbon-containing structure.

The subunits of Formulas II, VI and VII may comprise more than the two shown phosphorus groups of the phosphazene chain. For instance, the subunit comprising Formula VII may correspond to Formula VIII.

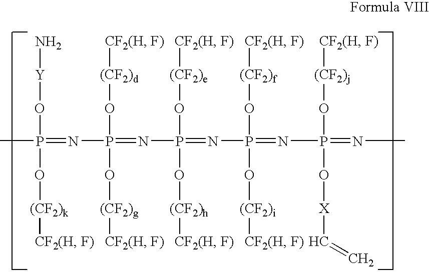

In Formula VIII, d, e, f, g, h and i are integers of from 1 to 16, which may be the same as one another or different. Also, the integers d, e, f, g, h and i may be the same as j and k or different.

Treatment of a substrate surface with material comprising the subunit of Formula II forms a polymeric coating which comprises a subunit which includes Formula IX (shown below). The polymeric coating can at least partially coat a substrate, and in some embodiments will coat all exposed surfaces of a substrate. The treatment to form the polymeric coating may comprise exposure of a surface to the material comprising Formula II, followed by curing of such material to form the crosslinked polymeric coating.

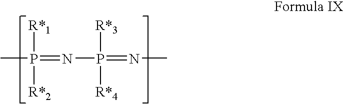

In Formula IX, R*₁, R*₂, R*₃ and R*₄ are carbon-containing groups which may be the same as R₁, R₂, R₃ and R₄, respectively, of Formula II, or which may be modified relative to such groups of Formula II. For instance, the one or more of R₁, R₂, R₃ and R₄ of Formula II that comprised an ω-olefin may be modified in formula IX to form a crosslink between polymeric chains of the coating. Also, any of R₁, R₂, R₃ and R₄ that contained a terminal amine (or other reactive group) may be modified to form a covalent bond to a substrate surface, or to from a crosslink between polymeric chains of the coating. For instance, if Formula II corresponds to Formula III, the polymeric coating may comprise a subunit which includes Formula X.

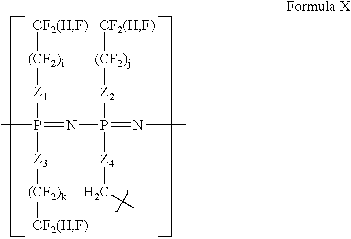

Formula X is identical to Formula III, except that the ω-olefin has opened to form a bond. Such bond may be a crosslink between polymeric chains, or may be a covalent attachment to a substrate.

As another example, if Formula II corresponds to Formula IV, the polymeric coating may comprise a subunit which includes Formula XI.

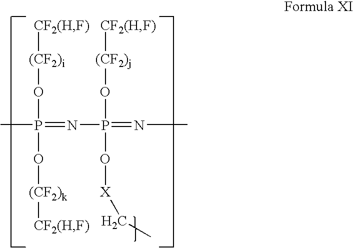

Formula XI is identical to Formula IV, except that the ω-olefin has opened to form a bond.

As another example, if Formula II corresponds to Formula V, the polymeric coating may comprise a subunit which includes Formula XII.

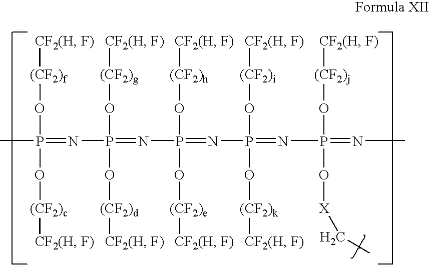

Formula XII is identical to Formula V, except that the ω-olefin has opened to form a bond.

As another example, if Formula II corresponds to Formula VI, the polymeric coating may comprise a subunit which includes Formula XIII.

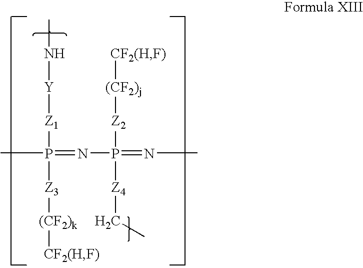

Formula XIII is identical to Formula VI, except that the ω-olefin has opened to form a bond, and the amine is forming a bond. The bond from the amine may be a covalent attachment to a substrate or a crosslink to another phosphazene polymer chain; and the bond from the opened ω-olefin may be a covalent attachment to a substrate or a crosslink to another phosphazene polymer chain. In some embodiments, the bond from the amine forms a covalent attachment to a polymeric surface composition of a substrate (such as a urethane-containing surface of a substrate), and the opened ω-olefin forms a crosslink to another phosphazene polymer chain.

As another example, if Formula II corresponds to Formula VII, the polymeric coating may comprise a subunit which includes Formula XIV.

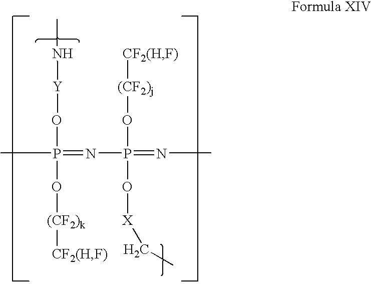

Formula XIV is identical to Formula VII, except that the ω-olefin has opened to form a bond, and the amine is forming a bond.

As another example, if Formula II corresponds to Formula VIII, the polymeric coating may comprise a subunit which includes Formula XV.

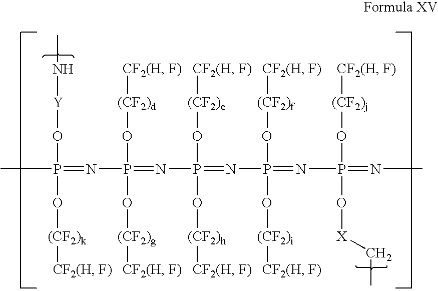

Formula XV is identical to Formula VIII, except that the ω-olefin has opened to form a bond, and the amine is forming a bond.

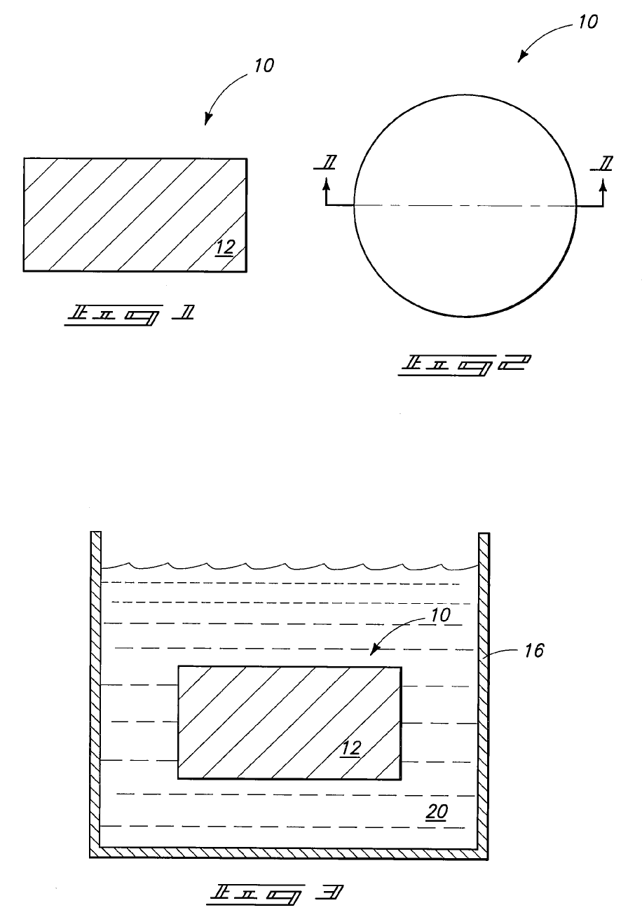

An example process for forming a phosphazene polymer coating around a substrate is described with reference to FIGS. 1-6.

Referring initially to FIGS. 1 and 2, a substrate 10 is illustrated. The substrate is shown to comprise a homogeneous composition 12. In other embodiments, the substrate may comprise multiple different layers. For instance, the substrate may comprise an internal composition, and an activated surface surrounding the internal composition.

The shown homogeneous composition of substrate 10 may correspond to any material, and may be, for example, ceramic, polymeric or metallic. In some embodiments, substrate 10 may be configured as a medical implant. In such embodiments, the substrate may, for example, comprise, consist essentially of, or consist of rigid metallic material, such as titanium; or may comprise, consist essentially of, or consist of flexible polymeric material, such as urethane. The substrate is shown having a disc shape. The substrate may have any configuration, and in other embodiments may be configured as, for example, a stent, artificial organ or organ part, artificial blood vessel, heart valve, skeletal support, or skeletal replacement.

Referring to FIG. 3, substrate 10 is placed within a vessel 16. The vessel has polymeric precursor 20 contained therein. The polymeric precursor may comprise any of the materials described above with reference to Formulas I-VIII, and is a liquid in the shown embodiment. The illustrated dipping of substrate 10 within the liquid precursor is one method for exposing surfaces of the substrate to the precursor. Any suitable method for such exposure may be utilized, and in other embodiments the exposure may be accomplished by, for example, spraying precursor across the surfaces. Although all of the surfaces of the substrate are shown exposed to the precursor, in other embodiments only some of the surfaces of the substrate may be exposed to the precursor.

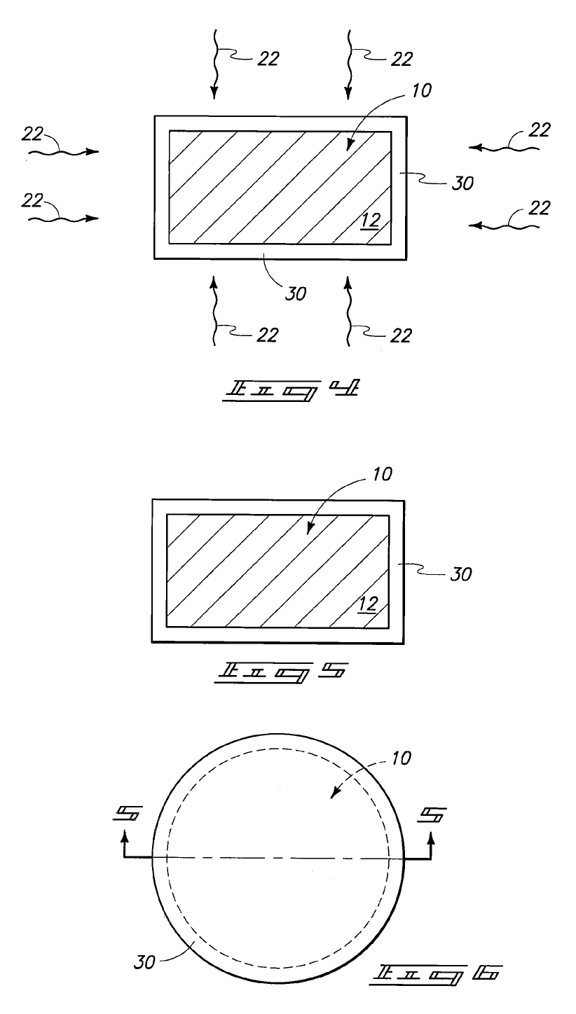

Referring to FIG. 4, substrate 10 is removed from vessel 16 (FIG. 3), and energy 22 is provided to polymerize material 20 (FIG. 3) associated with the substrate to convert the material to a polymerized coating 30. The energy 22 may be any form of energy suitable to cause polymerization of material 20, and may, for example, comprise electromagnetic radiation and/or thermal energy. If energy 22 comprises electromagnetic radiation, it may, for example, comprise UV light. In embodiments in which material 20 comprises one or more of the materials described above with reference to Formulas I-VIII, energy 22 may be thermal energy, and may correspond to heating of material 22 to a temperature of from about 110° C. to about 150° C., for a time of from about 10 minutes to about 120 minutes.

Although substrate 10 is shown removed from vessel 16 prior to the treatment with energy 22, in other embodiments the substrate may remain within the vessel during the treatment.

FIGS. 5 and 6 show substrate 10 at a processing stage subsequent to that of FIG. 4, and show the cured coating 30 surrounding an outer surface of substrate 10. An outer periphery of substrate 10 is indicated by a dashed line in the top view of FIG. 6, with the dashed line being utilized to indicate that substrate 10 is beneath material 30 in the shown view.

Coating 30 may be formed to any suitable thickness, and in some embodiments may be formed to a thickness of from about 0.1 micron to at least about 100 microns.

Coating 30 may be formed to have a flexibility which is at least that of substrate 10 so that coating 30 will flex with the substrate, rather than delaminating during flexure of the substrate. Flexibility of coating 30 may be adjusted through control of the amount of cross-linking within the coating and/or through control of the length of carbon chains utilized in the polymeric material.

In compliance with the statute, the subject matter disclosed herein has been described in language more or less specific as to structural and methodical features. It is to be understood, however, that the claims are not limited to the specific features shown and described, since the means herein disclosed comprise example embodiments. The claims are thus to be afforded full scope as literally worded, and to be appropriately interpreted in accordance with the doctrine of equivalents.

We claim:

1. A medical implant at least partially coated with polymeric material, the polymeric material comprising a subunit which includes: 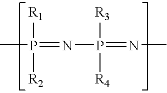
where R₁, R₂, R₃ and R₄ are carbon-containing groups, and where two or more of R₁, R₂, R₃ and R₄ may be the same as one another;
where at least one of R₁, R₂, R₃ and R₄ comprises —CF₂—CF₂(H,F); and
where at least one of R₁, R₂, R₃ and R₄ of one subunit is directly cross-linked to an R₁, R₂, R₃ or R₄ of another subunit.

2. The at least partially coated medical implant of claim 1 wherein at least one of R₁, R₂, R₃ and R₄ comprises an unsaturated hydrocarbon.

3. The at least partially coated medical implant of claim 1 wherein at least one of R₁, R₂, R₃ and R₄ comprises an ω-olefin.

4. The at least partially coated medical implant of claim 1 comprising a surface composition, and wherein at least one of R₁, R₂, R₃ and R₄ comprises an amine covalently bonded to the surface composition.

5. The at least partially coated medical implant of claim 1 comprising urethane, and wherein at least one of R₁, R₂, R₃ and R₄ comprises an amine covalently bonded to the urethane.

6. The at least partially coated medical implant of claim 1 wherein the subunit includes: 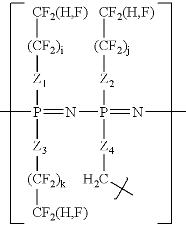
where Z₁, Z₂, Z₃, and Z₄ comprise N, S or O, and may be the same as one another or different; and
where i, j and k are integers of from 1 to 16, which may be the same as one another or different.

7. The at least partially coated medical implant of claim 6 wherein the subunit includes: 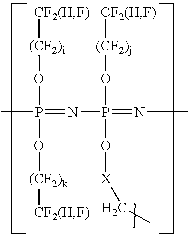
where X comprises one or more of carbon and heteroatoms.

8. The at least partially coated medical implant of claim 7 where X comprises a linear carbon-containing structure.

9. The at least partially coated medical implant of claim 7 where X comprises a cyclic carbon-containing structure.

10. The at least partially coated medical implant of claim 7 where X comprises an aromatic carbon-containing structure.

11. The at least partially coated medical implant of claim 1 wherein the subunit includes: 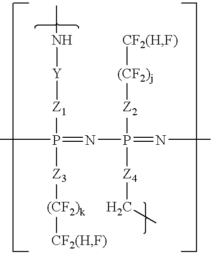
where Z₁, Z₂, Z₃, and Z₄ comprise N, S or O, and may be the same as one another or different;
where Y comprises one or more of carbon and heteroatoms; and
where j and k are integers of from 1 to 16, which may be the same as one another or different.

12. The at least partially coated medical implant of claim 11 wherein the subunit includes: 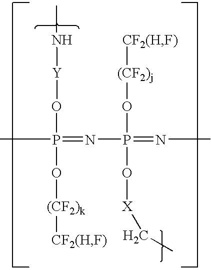
where X comprises one or more of carbon and heteroatoms.

13. The at least partially coated medical implant of claim 12 where X comprises a linear carbon-containing structure.

14. The at least partially coated medical implant of claim 12 where X comprises a cyclic carbon-containing structure.

15. The at least partially coated medical implant of claim 12 where X comprises an aromatic carbon-containing structure.

16. The at least partially coated medical implant of claim 12 where Y comprises a linear carbon-containing structure.

17. The at least partially coated medical implant of claim 12 where Y comprises a cyclic carbon-containing structure.

18. The at least partially coated medical implant of claim 12 where Y comprises an aromatic carbon-containing structure.

19. The at least partially coated medical implant of claim 1 wherein the subunit corresponds to: 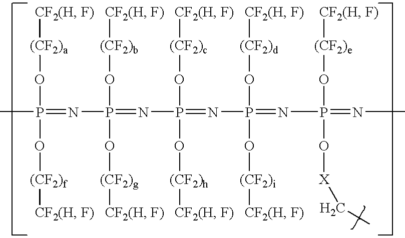
where X comprises one or more of carbon and heteroatoms; and
where a, b, c, d, e, f, g, h and i are integers of from 1 to 16, which may be the same as one another or different.

20. The at least partially coated medical implant of claim 19 where X comprises —((CH₂)₂O—)ₙ, where n is an integer from 1 to 16.

21. The at least partially coated medical implant of claim 1 wherein the subunit corresponds to: 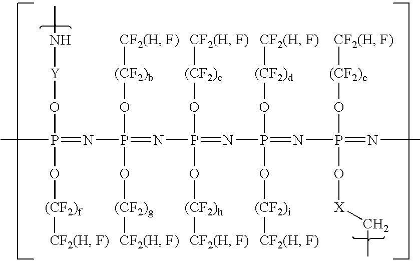
where X comprises one or more of carbon and heteroatoms;
where Y comprises one or more of carbon and heteroatoms; and
where b, c, d, e, f, g, h and i are integers of from 1 to 16, which may be the same as one another or different.

22. The at least partially coated medical implant of claim 21 where X comprises —((CH₂)₂O—)ₙ, where n is an integer from 1 to 16.

23. The at least partially coated medical implant of claim 21 where Y comprises —((CH₂)—)ₙ, where n is an integer from 1 to 16.

24. A method of coating a medical implant comprising:
exposing a surface of the implant to a composition comprising material containing a subunit which includes: 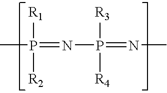
where R₁, R₂, R₃ and R₄ are carbon-containing groups; where at least one of R₁, R₂, R₃ and R₄ comprises —CF₂—CF₂(H,F); where two or more of R₁, R₂, R₃ and R₄ may be the same as one another; and where at least one of R₁, R₂, R₃ and R₄ comprises an ω-olefin; and
forming the composition into a polymeric coating adhered to the implant.

25. The method of claim 24 wherein the forming comprises covalently bonding the polymeric coating to said surface.

26. The method of claim 25 wherein the polymeric coating comprises linear chains of polymeric material; and
wherein the forming comprises cross-linking the linear chains to one another.

27. The method of claim 25 wherein at least one of R₁, R₂, R₃ and R₄ comprises an amine covalently bonded to the said surface.

28. The method of claim 25 wherein the surface comprises urethane, and wherein at least one of R₁, R₂, R₃ and R₄ comprises an amine covalently bonded to the urethane.

29. The method of claim 24 wherein the adhered polymeric coating is not covalently bonded to said surface.

30. The method of claim 29 wherein the surface comprises titanium.

31. The method of claim 24 wherein the subunit includes: 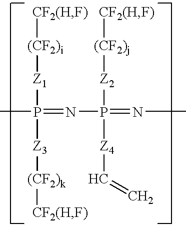
where Z₁, Z₂, Z₃, and Z₄ comprise N, S or O, and may be the same as one another or different; and
where i, j and k are integers of from 1 to 16, which may be the same as one another or different.

32. The method of claim 31 wherein the subunit includes: 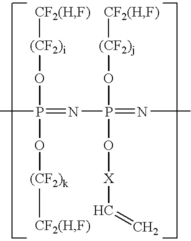
where X comprises one or more of carbon and heteroatoms.

33. The method of claim 32 where X comprises —((CH₂)₂O—)ₙ, where n is an integer from 1 to 16.

34. The method of claim 24 wherein the subunit includes: 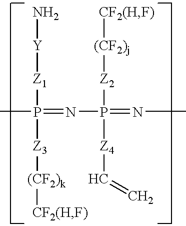
where Z₁, Z₂, Z₃, and Z₄ comprise N, S or O, and may be the same as one another or different;
where Y comprises one or more of carbon and heteroatoms; and
where j and k are integers of from 1 to 16, which may be the same as one another or different.

35. The method of claim 34 wherein the subunit includes: 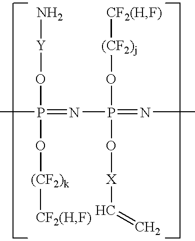
where X comprises one or more of carbon and heteroatoms.

36. The method of claim 35 where X comprises —((CH₂)₂O—)ₙ, where n is an integer from 1 to 16.

37. The method of claim 35 where Y comprises —((CH₂)—)ₙ, where n is an integer from 1 to 16.

38. The method of claim 24 wherein the subunit corresponds to: 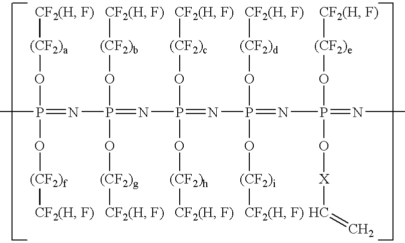
where X comprises one or more of carbon and heteroatoms; and
where a, b, c, d, e, f, g, h and i are integers of from 1 to 16, which may be the same as one another or different.

39. The method of claim 38 where X comprises —((CH₂)₂O—)ₙ, where n is an integer from 1 to 16.

40. The method of claim 24 wherein the subunit corresponds to: 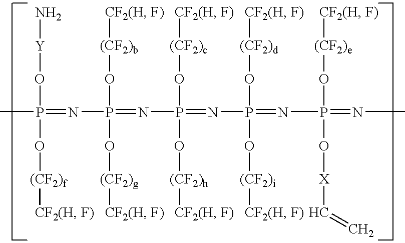
where X comprises one or more of carbon and heteroatoms;
where Y comprises one or more of carbon and heteroatoms; and
where b, c, d, e, f, g, h and i are integers of from 1 to 16, which may be the same as one another or different.

41. The method of claim 40 where X comprises —((CH₂)₂O—)ₙ, where n is an integer from 1 to 16.

42. The method of claim 40 where Y comprises —((CH₂)—)ₙ, where n is an integer from 1 to 16.

43. A method of coating a substrate, comprising:
exposing the substrate to a material containing a subunit of: 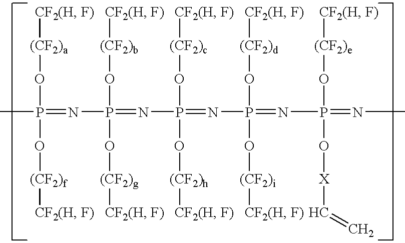
where X comprises one or more of carbon and heteroatoms; and where a, b, c, d, e, f, g, h and i are integers of from 1 to 16, which may be the same as one another or different; and
treating the material to convert the material to a polymeric coating surrounding at least a portion of the substrate.

44. The method of claim 43 where X comprises —((CH₂)₂O—)ₙ, where n is an integer from 1 to 16.

45. The method of claim 43 where the treatment comprises heating of the material to a temperature of at least about 110° C.

46. The method of claim 43 where the treatment comprises exposure to ultraviolet radiation.

47. The method of claim 43 where the substrate has a metallic outer surface.

48. The method of claim 43 where the substrate has a ceramic outer surface.

49. The method of claim 43 where the substrate has a polymeric outer surface.

50. The method of claim 43 where the substrate consists of titanium.

51. The method of claim 43 where X comprises —((CH₂)₂O—)ₙ, where n is an integer from 1 to 16.

52. The method of claim 43 where Y comprises —((CH₂)—)ₙ, where n is an integer from 1 to 16.

53. A method of coating a substrate, comprising:
exposing the substrate to a material containing a subunit of: 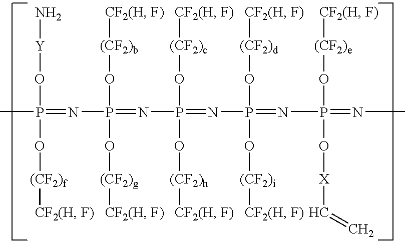
where X comprises one or more of carbon and heteroatoms; where Y comprises one or more of carbon and heteroatoms; and where b, c, d, e, f, g, h and i are integers of from 1 to 16, which may be the same as one another or different; and
treating the material to convert the material to a polymeric coating surrounding at least a portion of the substrate.

54. The method of claim 53 where the treatment comprises heating of the material to a temperature of at least about 110° C.

55. The method of claim 53 where the treatment comprises exposure to ultraviolet radiation.

56. The method of claim 53 where the substrate has a polymeric outer surface.

57. The method of claim 53 where the substrate outer surface comprises urethane.
